(19) United States
(12) Patent Application Publication
(10) Pub. No.: US 2006/0224151 A1
(43) Pub. Date: Oct. 5, 2006

(54) System and method for projecting a virtual user interface for controlling electrosurgical generator

(75) Inventor: Luke Waaler, Fort Collins, CO (US)

Correspondence Address: UNITED STATES SURGICAL,;A DIVISION OF TYCO HEALTHCARE GROUP LP, 195 MCDERMOTT ROAD, NORTH HAVEN, CT, 06473, US

(73) Assignee: SHERWOOD SERVICES AG

(21) Appl. No.: 11/388,303

(22) Filed: Mar. 24, 2006

Related U.S. Application Data
(60) Provisional application No. 60/666,848, filed on Mar. 31, 2005.

(51) Int. Cl. A61B 18/18

(52) U.S. Cl. 606/34

(57) ABSTRACT

A system and method for projecting a virtual user interface for controlling an electrosurgical generator is disclosed. The system includes a generator for supplying RF energy to an active electrode, and an interface device adapted to project a virtual interface and infrared light onto a surface in proximity to a surgical site and to detect at least one input within the interface. The interface device further adapted to correlate the at least one input with a corresponding command and transmit the command to the generator, for adjusting the RF energy intensity.

### PRIORITY CLAIM

This patent claims priority to a U.S. Provisional Application Ser. No. 60/666,848 entitled “System and Method for Projecting a Virtual User Interface for Controlling Electrosurgical Generator” filed on Mar. 31, 2005 by Luke Waaler, the entire contents of which are hereby incorporated by reference herein.

### BACKGROUND

Technical Field

The present disclosure relates generally to an apparatus and method for controlling an electrosurgical generator, and more particularly, to a system and method for controlling energy output of the generator using a virtual user interface projected to a surface near or at the surgical site.

Background of Related Art

During electrosurgery, a source or active electrode delivers energy, such as radio frequency (RF) energy, from an electrosurgical generator to a patient and a return electrode (or a plurality thereof) carries current back to the electrosurgical generator. In monopolar electrosurgery, the source electrode is typically a hand-held instrument placed by the surgeon at the surgical site and the high current density flow at this electrode creates the desired surgical effect of ablating, cutting or coagulating tissue. The patient return electrodes are placed at a remote site from the source electrode and are typically in the form of pads adhesively fixed to the patient.

Generally, the output of the electrosurgical energy is controlled using input devices such as switches, intensity control knobs, touchscreen, etc., disposed on the generator. The surgeon adjusts the RF energy depending on the procedure. For instance, a low voltage is used to perform cutting procedures, this generates the least amount of heat and results in the least amount of hemostasis to surrounding tissue. A high voltage is best suited for coagulating tissue, however, it produces high heat with high hemostatis. Thus, a surgeon needs to continually monitor the effects of the electrosurgical procedure and adjust the RF energy output accordingly.

Rapid adjustment of RF energy output levels is especially important in medical procedures where little or no anesthesia is used. In such procedures, the surgeon needs to be even more mindful of the effects the procedure is having on the patient, e.g., a comfort level of the patient, and adjust the RF energy if the electrosurgical procedure is causing undesirable effects. However, conventional electrosurgical systems, which place RF energy controls at the generator, require that the surgeon momentarily stop the procedure and focus his attention away from the surgical site to adjust the intensity of the RF energy. The surgeon may adjust the current by moving over to the generator or ask for assistance to do so. These methods have drawbacks, for example, asking another person to adjust the current does not provide the same level of exactitude and feedback that the surgeon would be able to achieve in adjusting the RF energy individually. Further, any adjustment by the surgeon would require that the surgeon leave the sterile field and cease the procedure temporarily, resulting in unneeded interruptions and risks of contamination at the surgical site.

Therefore, there is a need for systems and methods to allow the surgeon to control RF energy output during electrosurgical procedures at the surgical site.

### SUMMARY

The present disclosure provides for an interface device to project a virtual user interface near a surgical site during electrosurgery to control an electrosurgical generator. More specifically, the interface is adapted to adjust the intensity of radiofrequency energy outputted by the generator. The interface device includes a pattern projector to display an interface, an infrared light source to illuminate the interface and thereby detect user input. The interface also includes a sensor module adapted to detect user input when objects pass through the infrared light at predetermined regions. The interface device analyzes detected input commands to extrapolate the commands and transmits them to the generator which thereafter adjusts the energy output accordingly.

According to one embodiment of the present disclosure, a system for projecting a virtual user interface for controlling intensity of radio frequency (RF) energy is disclosed. The system includes a generator for supplying RF energy to an active electrode, and an interface device adapted to project a virtual interface and infrared light onto a surface in proximity to a surgical site and to detect at least one input within the interface. The interface device further adapted to correlate the at least one input with a corresponding command and transmit the command to the generator, for adjusting the RF energy intensity.

According to another embodiment of the present disclose, a method for projecting a virtual user interface which controls an electrosurgical generator is disclosed. The method includes the step of providing and a virtual interface device. The interface device adapted to project a virtual interface and infrared light onto a surface in proximity to a surgical site and to detect at least one input within the interface. The interface device further adapted to correlate the at least one input with a corresponding command and transmit the command to the generator, for adjusting the RF energy intensity. The method also includes the steps of projecting an interface onto a surface in proximity to a surgical site, projecting infrared light onto the surface, detecting an input within the virtual interface, correlating the input with a corresponding command, and transmitting the command to a generator, wherein the generator adjusts radio frequency energy intensity.

According to a further aspect of the present disclosure a system for controlling a generator is disclosed. The system includes a generator which supplies RF energy and an interface device which is adapted to project a virtual interface onto a surface in proximity to a surgical site. The interface device includes a sensor which detects an input. The interface device is also configured to correlate the input with a corresponding command and transmit the command to the generator for adjusting one or more parameters associated with the generator. The parameter may include power, current, intensity, gas flow, frequency, fluid flow, vibration, resonance and temperature.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a schematic illustration of a monopolar electrosurgical system;

FIG. 2 is a schematic illustration of a virtual user interface device according to the present disclosure; and

FIG. 3 shows a method for controlling an electrosurgical generator through the interface device of FIG. 2.

### DETAILED DESCRIPTION

Embodiments of the present disclosure are described below with reference to the accompanying drawings. In the following description, well-known functions or constructions are not described in detail to avoid obscuring the present disclosure in unnecessary detail. Although the foregoing disclosure describes embodiments with reference to a monopolar ablation instrument, those skilled in the art will understand that the principles of the present disclosure can be utilized in a variety of electrosurgical systems (e.g., bipolar).

The present disclosure provides for a device for projecting a virtual user interface at or near a surgical site for controlling an electrosurgical generator. In particular, the interface controls the intensity of the RF energy supplied by the generator. The interface device projects a virtual interface near a surgical site as well as infrared light which is used in detecting motion at the interface. The interface device detects input motions and correlates the input motions with control commands which are then relayed to the generator, where the intensity of the RF energy is adjusted accordingly.

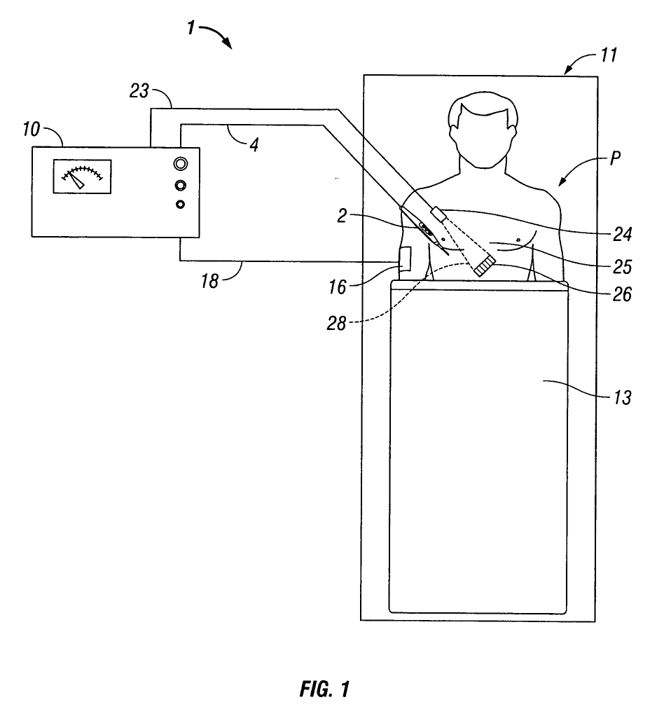

FIG. 1 is a schematic illustration of a monopolar electrosurgical system 1. The system 1 includes a surgical instrument 2, e.g., an active electrode, for treating tissue at a surgical site 25. The patient P is shown lying on a surgical table 11 and covered with a sterile cover 13. Electrosurgical energy is supplied to the instrument 2 by a generator 10 via a cable 4 allowing the instrument 2 to ablate, cut, coagulate or otherwise treat tissue. The electrosurgical system 1 also includes a return electrode 16 placed on an arm of the patient to return energy from the patient to the generator 10 via a wire 18. It should be appreciated that the system 1 may include a plurality of return electrodes that are placed on any part of the body which provides for maximum electrical conductivity (e.g., muscle tissue). The return electrode 16 may include a split pad which is adhesively attached to the patient's skin.

The system 1 also includes a virtual user interface device 24 positioned near the surgical site 25 to project a virtual interface 26 thereto. The interface device 24 is preferably attached via a support platform or bracket (not shown) to the surgical table 11 or another stablizing fixture (e.g., operating overhead lamp fixture). The interface device 24 projects the virtual user interface 26 near or at the surgical site 25 and reads input motions using infrared (IR) beams 28 projected at the same area. The interface 26 may be projected on any surface near the surgical site 25 (e.g., the sterile cover 18, a stainless steel tray, the patient P, etc.). The interface device 24 is in electrical communication with the generator 10 through wire 23 which allows the interface device 24 to control the intensity of the RF energy output as well as other functions of the generator 10.

Projecting the interface 26 for controlling the intensity of the RF energy near or at the surgical site allows the surgeon to adjust the current without leaving the sterile field of the surgical table 11 as well as provides for a quicker response in lowering current in case of an emergency.

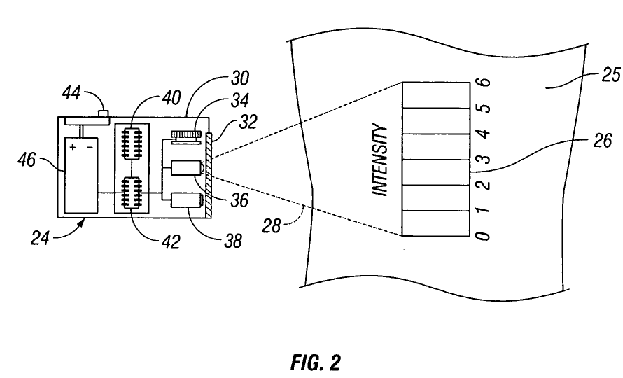

FIG. 2 shows a more detailed view of the interface device 24, which includes a pattern projector 38 for projecting the interface 26, an IR light source 36 for projecting the IR beams 28, and a sensor module 34 for detecting user input. Components which perform substantially the same functions are available from Canesta Inc., located in Sunnyvale, Calif., as part of Canesta Keyboard™ Perception Chipset™: Canesta Keyboard Sensor Module (SM-CK100), Canesta Keyboard Light Source (IR-CK100), and Canesta Keyboard Pattern Projector (PP-CK100).

The pattern projector 38, the IR light source 36, and the sensor module 34 are controlled by electronic circuitry such as a microprocessor 42 which may receive instructions from a memory module 40. The components of the interface device 24 are powered using a power source 46 which may be an independent battery or a parasitic device withdrawing power from the RF energy transmitted to the instrument 2 through the wire 4 using a transformer (not shown). A switch 44 is used to toggle the interface device 24 on and off. In addition, the switch 44 may be used to control the generator 10 (e.g., toggle it on and off). The components of the interface device 24 are enclosed in a housing 30 having a transparent front wall 32.

With reference to FIG. 2, the pattern projector 38 projects the interface 26 having a scale, a label, e.g., “Intensity,” and numbers, e.g., “0” through “6”, indicating the intensity level for controlling the intensity of the RF energy. The interface 26 displays selected input parameters so as not to clutter the interface with other inputs. It is envisioned that the interface 26 may display other input means, such as buttons (e.g., numeric keypad), sliding scales, pointer pads, arrows including current value centered therein, etc. The type of interface may be determined by the particular instrument being used or may be selectable at the generator 10 by the user.

The pattern projector 38 includes a wide angle lens, a light source, and a projection pattern (not shown). The light source illuminates the projection pattern through a wide angle lens increasing the pattern size as it is projected to display the interface 26. The projection pattern may be a slide having a desired interface projected thereon. The projection pattern may be interchangeable so that a specific pattern having a scale more suitable for a particular procedure may be projected instead of the interface 26 (e.g., a projection representing a more sensitive scale displaying more intensity levels).

Those skilled in the art will appreciate that the interface device may also output other data such as temperature and impedance values at the surgical site 25, alarm conditions, etc. This would allow the surgeon to maintain his visual focus near or at the surgical site.

The IR source 36 projects the dispersed IR beams 28 to overlap the entire area where the interface 26 is projected. The IR beams 28 allows the sensor module 34 to read inputs by detecting any object that is placed at the interface 26 and interrupts the IR beams 28 (e.g., a finger). The sensor module 34 detects the objects entering the area of the interface 26 by detecting the disruptions in the IR beams 28. Thus, when a finger is touching a particular area of the interface 26, the IR beams 28 projected by the IR source 36 is interrupted and the sensor module 34 detects the finger at that particular location. The sensor module 34 thereafter transmits the location where input/interruption occurred to the microprocessor 42 which matches the position of the input signal with a corresponding input command and transmits the corresponding command to the generator 10.

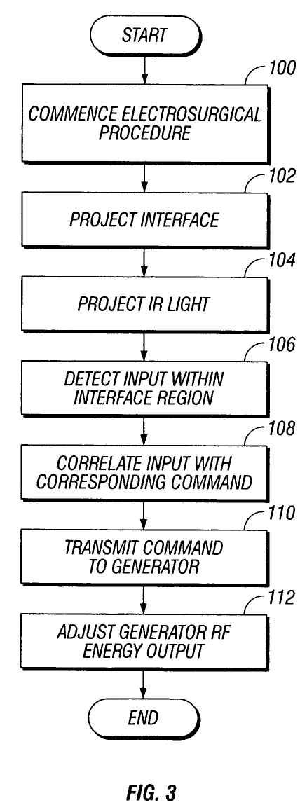

FIG. 3 shows a method for controlling intensity of the electrosurgical generator 10 using the interface device 24. In step 100, the electrosurgical procedure is commenced, the patient is laid out on the table 11 and covered with the sterile cover 13. The generator 10 is activated either using the switch 44 positioned on the interface device 24 or using a similar switch located on the generator 10. As discussed above, the majority of controls for the generator 10, except for the intensity controls, may remain at the generator 10. In addition, the interface device 24 is activated.

In step 102, the interface device 24 projects the interface 26 through the pattern projector 38 onto the surgical site 25 or the sterile cover 13 so that the interface 26 provides easy access to at least one controllable variable. The interface device 24 also projects the IR beams 28 to the interface 26 using the IR source 36 in step 104. This sets up the interface 26 for input and the interface device 24 is ready to accept input commands.

In the present embodiment, the surgeon controls the intensity of the RF energy using the interface 26 by placing a finger or another object next to the corresponding desired level (e.g., 0 through 6) of the interface 26. The interface device 24 reads the input commands by detecting disruptions in the IR beams 28 through the sensor module 34 in step 106. The sensor module 34 transmits the input signals to the microprocessor 42 which, in step 108, correlates the input signal with a corresponding command. The microprocessor 42, in step 110, transmits the corresponding command to the generator 10, where in step 112 the generator adjusts the RF energy output accordingly.

For instance, a surgeon that wishes to input an intensity level of “4” would place a finger in that region of the interface 26. The sensor module 34 would detect the disturbance in that region of the IR beams 28 and would transmit that signal to the microprocessor 42. The microprocessor 42 is aware that the input signal from that region corresponds to a command to set the intensity level to “4” and transmits that command to the generator, which then adjusts the RF energy output to intensity level “4.”

The present disclosure allows a surgeon to control intensity of RF energy supplied to the surgical site without leaving the sterile field and without diverting his attention from the surgical site. This minimizes the risk of introducing infection to the site and allows the surgeon to adjust the RF energy almost immediately as a need arises. Such rapid energy adjustment is especially important where the patient is undergoing an electrosurgical procedure without anesthesia and the surgeon needs to control the level of pain and discomfort the patient is experiencing.

A method for projecting a virtual user interface which controls an electrosurgical generator is disclosed. The method includes the step of providing and a virtual interface device. The interface device adapted to project a virtual interface and infrared light onto a surface in proximity to a surgical site and to detect at least one input within the interface. The interface device further adapted to correlate the at least one input with a corresponding command and transmit the command to the generator, for adjusting the RF energy intensity. The method also includes the steps of projecting an interface onto a surface in proximity to a surgical site, projecting infrared light onto the surface, detecting an input within the virtual interface, correlating the input with a corresponding command, and transmitting the command to a generator, wherein the generator adjusts radio frequency energy intensity.

The described embodiments of the present disclosure are intended to be illustrative rather than restrictive, and are not intended to represent every embodiment of the present disclosure. For example, it is envisioned that the virtual interface may be utilized with many different types of instruments including electrosurgical pencils, gas coagulators, ultrasonic instruments, resistively heated instruments, ablation instruments, etc. The virtual interface may control any number of different parameters associated with each of these aforementioned instruments including: modality, gas flow, coolant fluid flow, resonance, frequency, temperature, power, current, vibration, etc. Moreover, the interface device may be arranged to project more than one virtual interface onto the surface for controlling more than one parameter. The interface device may also be configured to allow the surgeon to selectively control one or more operating parameters by a user interface attached to the interface device or the generator. Still further, the user interface may be configured to also project patient vitals next to the virtual interface to allow quick patient monitoring by the surgeon, e.g., patient temp, heart data, oxygen level, etc.

Various modifications and variations can be made without departing from the spirit or scope of the disclosure as set forth in the following claims both literally and in equivalents recognized in law.

What is claimed is:

1. A system for projecting a virtual user interface for controlling of radio frequency (RF) energy intensity, the system comprising:
a generator that supplies RF energy to an active electrode; and
an interface device adapted to project a virtual interface onto a surface in proximity to a surgical site, said interface device including at least one sensor that detects at least one input, the interface device being configured to correlate the at least one input with a corresponding command and transmit the command to the generator, wherein for adjusting RF energy intensity.

2. A system as in claim 1, wherein the interface device includes a pattern projector that projects the interface onto the surface.

3. A system as in claim 1, wherein the interface device includes an infrared light source that projects infrared light onto the surface.

4. A system as in claim 3, wherein the interface device includes a sensor module that detects the at least one input motion within the interface.

5. A system as in claim 1, wherein the interface is a scale representing RF energy intensity.

6. A method for projecting a virtual user interface that controls an electrosurgical generator, the method comprising the steps of:
providing an interface device adapted to project a virtual interface onto a surface in proximity to a surgical site and to detect at least one input within the virtual interface, the interface device being configured to correlate the at least one input with a corresponding command and transmit the command to an electrosurgical generator for adjusting RF energy intensity;
projecting a virtual interface onto a surface in proximity to a surgical site;
projecting infrared light onto the surface;
detecting at least one input within the virtual interface;
correlating the at least one input with a corresponding command; and
transmitting the command to the electrosurgical generator, for adjusting RF energy intensity.

7. A method as in claim 6, wherein a pattern projector projects the virtual interface onto the surface.

8. A method as in claim 6, wherein an infrared light source projects infrared light onto the surface.

9. A method as in claim 6, wherein a sensor module detects the at least one input within the virtual interface.

10. A method as in claim 6, wherein the virtual interface is a scale representing RF energy intensity.

11. A system for controlling a generator, comprising:
a generator which supplies RF energy;
an interface device adapted to project a virtual interface onto a surface in proximity to a surgical site, said interface device including at least one sensor which detects at least one input, said interface device being configured to correlate the at least one input with a corresponding command and transmit the command to the generator for adjusting at least one parameter associated with the generator, the at least one parameter including at least one of power, current, intensity, gas flow, frequency, fluid flow, vibration, resonance and temperature.
